(19) United States
(12) Patent Application Publication
(10) Pub. No.: US 2006/0205090 A1
(43) Pub. Date: Sep. 14, 2006

(54) Water-soluble conjugates for electrochemical detection

(76) Inventors: Michael W. Newton, San Diego, CA (US); Charles Ajith Wijayawardhana, San Diego, CA (US)

Correspondence Address: AZURE INSTITUTE;INTELLECTUAL PROPERTY DEPT., 4108 SORRENTO VALLEY BOULEVARD, SAN DIEGO, CA, 92124, US

(21) Appl. No.: 11/081,001

(22) Filed: Mar. 14, 2005

(51) Int. Cl. G01N 33/543

(52) U.S. Cl. 436/518

(57) ABSTRACT

The present invention provides water-soluble conjugates and methods of using them in diagnostic and detection assays. Devices for performing detection and quantitation assays are also provided. In various embodiments the conjugates are useful in immunoassays and later flow assays. The invention provides methods of preparing the conjugates that result in higher yields and higher sensitivities for the assays. The invention also provides water-soluble conjugates utilizing electrochemical signal components capable of detecting analytes with very high sensitivity.

The present application is a continuation-in-part of U.S. Ser. No. 10/924,738, filed Aug. 23, 2004, which is hereby incorporated by reference in its entirety, including all Tables, Figures, and Claims.

### FIELD OF THE INVENTION

The present invention relates to compositions of water-soluble conjugates useful in diagnostic assays, methods for preparing and using the conjugates, immunoassays, lateral flow assays, and test devices.

### BACKGROUND OF THE INVENTION

There is a continuing need for superior methods of preparing conjugates which exhibit a high degree of sensitivity and specificity when employed in immunochemical assays, such as home pregnancy and fertility tests.

Various strategies for improving the sensitivity and reliability of immunoassays have been reviewed by L. J. Kricka (1994) Clin. Chem. 40, 347-357.

EP 0 594 772 B1 relates to water-soluble, polymer-based conjugates comprising moieties derived from divinyl sulfone. EP 0 594 772 B1 describes the possibility of enhancing the attachment of molecular species, such as antibodies and antigens, to a water-soluble carrier molecule by taking advantage of the so-called “salting out” effect. It turned out, however, that by increasing the salt concentration to about 1 M an irreversible precipitate was formed.

U.S. Pat. No. 6,627,460 to Lihme et al., provides methods of water-soluble cross linked conjugates, and methods of their use. The patent provides methods of further increasing the concentration of salt in the reaction mixture, which causes a reversible (i.e. a re-dissolvable) precipitate to form containing a water-soluble conjugate, which is useful in various immunochemical assays such as in lateral flow devices.

### SUMMARY OF THE INVENTION

The present invention provides compositions of water-soluble conjugates for use in diagnostic and detection assays, and methods of their preparation and use. In various embodiments the conjugates are useful in immunoassays and lateral flow assays. The invention provides methods of preparing the conjugates that result in higher yields in the preparation of the conjugates, and higher sensitivities in the assays. The invention also provides water-soluble conjugates that can be produced more economically. The invention also provides devices for use in conducting detection and quantitation assays for a variety of ligands of interest. The invention also provides water-soluble conjugates that utilize electrochemical detection methods and have very high sensitivity.

Methods of preparing water-soluble conjugates are discussed in U.S. Pat. No. 6,627,460, which is hereby incorporated by reference in its entirety, including all tables, figures, and claims. These methods generally involve the preparation of a water-soluble conjugate having a carrier component, a linking component, a spacer component, a signal component and a targeting element for a ligand to be detected or a ligand to be detected (a primary targeting component). The signal component is covalently attached to the spacer component and the spacer component is covalently attached, via the linking component, to the carrier component. The methods involve a) reacting a water-soluble intermediate conjugate having a carrier component, a linking component, a spacer component, and a signal component (the signal component being covalently attached to the spacer component and the spacer component being covalently attached, via the linking component, to the carrier component), with at least one primary targeting component (a targeting element for a ligand to be detected or ligand to be detected). The reaction occurs with unreacted reactive moieties derived from the linking component, in an aqueous solution. The conditions are such that a reversible precipitate is formed. The reversible precipitate containing the water-soluble conjugate is re-dissolved in an aqueous medium; and c) optionally, the water-soluble cross-linked conjugate is subjected to a purification step. Further details of the reaction parameters are provided in U.S. Pat. No. 6,627,460, which is hereby incorporated by reference in its entirety, including all tables, figures, and claims. In various embodiments the conjugates can be cross-linked to one another to form larger conjugate molecules.

While examples of the arrangement of the water-soluble conjugates are provided herein, other arrangements are possible. For example, the targeting element can be attached to the carrier via the linker, or can be attached to the spacer or to a non-specific protein, as described below. Also, the signal component can be attached to the carrier, or to the spacer, or even to the targeting element. The precise arrangement of components can be varied in any manner to result in a water-soluble conjugate that functions as a reagent and the assay is performed and provides a useful result.

In a first aspect the invention provides methods for preparing a water-soluble conjugate involving a) preparing a water-soluble conjugate having at least one carrier, at least one linker, at least one signal component, and at least one targeting element for a ligand to be detected or a ligand to be detected, as a reversible precipitate in a suspension. The suspension is subjected to sonication to form a sonicated formulation, and a supernatant is separated from the formulation containing the water-soluble conjugate. Optionally, the water-soluble conjugate can be purified from the supernatant. In one embodiment the water-soluble conjugate is purified by gel filtration (or size exclusion) chromatography. In one embodiment the gel filtration chromatography is performed using a medium with an average size exclusion of 300 kD.

In various embodiments of the inventions described herein, the water-soluble conjugate can also contain a spacer component. In one embodiment the carrier is covalently attached to the linker and the signal component is covalently attached to the spacer. The water-soluble conjugate can be prepared by contacting a water-soluble intermediate conjugate with the ligand to be detected or with the targeting element for a ligand in the presence of a lyotropic salt at a concentration of at least about 1.25 M. In other embodiments the concentration of the lyotropic salt can be at least about 1.5 M or at least about 1.75 M or at least about 2.0 M or at least about 2.5 M. A “water-soluble conjugate” contains a carrier, a linker, a targeting element for a ligand to be detected or a ligand to be detected, a signal component, and can optionally also contain a spacer component. By “water-soluble intermediate conjugate” is meant a molecule containing a carrier, a linker, and a signal component. A water-soluble intermediate conjugate may also contain a spacer component. By “water-soluble intermediate precursor” is meant a molecule having any two or more of the components of a water-soluble conjugate and that is not a water-soluble conjugate or intermediate conjugate. In one embodiment the water soluble intermediate precursor contains a carrier and a linker. In another embodiment the precursor contains the carrier, linker, and spacer component. In one embodiment the water-soluble intermediate conjugate contains the carrier, linker, signal component, and spacer components. “Sonication” refers to the known technique used in chemistry and biology of exposure to a high frequency sound energy. It is also sometimes referred to as ultrasonication. The sonication can performed at any appropriate power, e.g., at least about 300 watts, or at least about 500 watts, or at least about 700 watts, or at least about 900 watts, or at least about 1000 watts, or at greater than 1000 watts. Any desirable frequency can also be used such as (for example) from 20 to 24 kHz. As used herein, “about” means plus or minus 10%. The term “reversible precipitate” indicates that the precipitate formed is capable of being re-dissolved upon dilution with aqueous solution at 25° C.

The lyotropic salt can contain components such as sulphates, phosphates, citrates and tartrates of lithium, sodium, potassium, calcium and ammonium, and can be present at a concentration of about 2.5 M. In one embodiment the salt is potassium phosphate or sodium phosphate.

In the present context the term “water soluble” when used in connection with the conjugates means that the conjugates obtained should be soluble in an aqueous medium, such as water, at room temperature, i.e. the cross-linked conjugates obtained by the methods disclosed herein should give rise to a solution which is substantially clear and homogeneous as judged by visual inspection of the sample.

In various embodiments the conjugates obtained have a water solubility of at least 0.1, or at least 0.2 or at least 0.5, or at least 1, or at least 3, or at least 5, or at least 7, or from 5 to 10, or from 4 to 11, or at least 10, or at least 20, or at least 30, or at least 40, or at least 50, or at least 100, and in particular at least 200 mg dry conjugate per ml water at 25° C. The present invention also provides water-soluble conjugate prepared according to any method of the invention.

In another aspect the present invention provides methods of preparing a water-soluble conjugate involving preparing a water-soluble conjugate as described herein as a precipitate in a suspension. A pellet containing the water-soluble conjugate is separated from the suspension, and the pellet washed with an aqueous solution to form a second suspension. A pellet is separated from the second suspension containing the water-soluble conjugate. The water-soluble conjugates prepared according to these methods can have the same structure as those conjugates described herein. For example, the conjugates can further contain a spacer, and the carrier can be covalently attached to the linker, and the signal component covalently attached to the spacer.

In one embodiment the water-soluble conjugate is purified by the process of separating the precipitate from a supernatant, forming a suspension of the precipitate in an aqueous solution, and separating the precipitate from a supernatant. The conjugates can contain a non-specific protein attached to the carrier via the linker (e.g., bovine serum albumin, an immunoglobulin). In one embodiment the targeting element is an antibody that has been treated with a reducing agent. By “reducing agent” is meant a substance that chemically reduces other substances by donating an electron or electrons. Examples of reducing agents include beta-mercaptoethanol, dithiothreitol, and 2-iminothiolane. By “washing the pellet” is meant that the pellet is placed into contact with the aqueous solution and agitated. The agitation can be by any method, for example by vortexing or stirring or shaking the container. Portions of the pellet may break off of the initial pellet during the agitation, and can be re-pelleted by centrifugation or another method. In another embodiment no further purification step is performed on the water-soluble conjugate after the washing with the aqueous solution.

In one embodiment the water-soluble conjugate is separated from the supernatant by centrifugation, although centrifugation is not necessary to practicing the method. The conjugate can also be purified from the supernatant by any convenient techniques, such as by gel filtration. By “supernatant” is meant the liquid portion of a sample.

In another aspect, the present invention provides water-soluble conjugates containing a carrier, a linker covalently bound to the carrier, a signal component, a targeting element for a ligand to be detected or a ligand to be detected, and a non-specific protein. In one embodiment the non-specific protein is covalently bound to the carrier via the linker. In another embodiment the non-specific protein is bound to the carrier via the linker and to no other component (other than the linker) of the conjugate. In other embodiments at least 2% or at least 3% or at least 5% or at least 10% or at least 15% or at least 20% of the non-specific proteins are bound to the carrier via the linkers and to no other component (other than the linker) of the conjugate. In other embodiments at least 50% or at least 60% or at least 70% or at least 75% or at least 80% or at least 85% or at least 90% or at least 95% of the non-specific proteins are bound to the carrier via the linker and to no other component (other than the linker) of the conjugate. In related embodiments all the same percentages of linkers recited above are bound to a non-specific protein, and the non-specific protein is bound to the linker and to no other component of the water-soluble conjugate. Any of the water-soluble conjugates can also contain a spacer component.

The “non-specific protein” is a protein that does not have a binding specificity or target within the context that it is used. The non-specific protein is typically linked to the water-soluble conjugate by a linking chemistry. Bovine serum albumin, immunoglobulins, keyhole limpet hemocyanin, and other proteins are examples of non-specific proteins. In one embodiment the non-specific protein is a protein other than the one used as a spacer (when a spacer is present), but the spacer and non-specific protein can also be the same protein used to accomplish different functions. The non-specific protein can contain amino groups, which are used to covalently bind the non-specific protein to the conjugate, although other suitable linking chemistries may also be used. In another embodiment, the spacer and the non-specific protein are independently and covalently attached to the carrier via the linker; the signal component is covalently attached to the spacer; and the ligand to be detected or targeting element for a ligand to be detected is covalently attached to the carrier.

In other embodiments the signal component is attached to either or both of the spacer and/or the non-specific protein. It is also possible to attach the non-specific protein and spacer to the carrier via the linker, and attach the targeting element or ligand, and signal component to either or both of the non-specific protein and spacer.

In another aspect, the present invention provides methods for preparing a water-soluble conjugate involving a) contacting a water-soluble intermediate conjugate having a carrier, a linker, a spacer, and a signal component, with i) a targeting element for a ligand to be detected or ii) a ligand to be detected, to form a suspension containing a precipitate comprising the water-soluble conjugate. The method also involves extracting the water-soluble conjugate from the suspension. The targeting element for a ligand to be detected or the ligand to be detected is pre-treated with a reducing agent prior to contact with the water-soluble intermediate conjugate. The extraction can be performed by any suitable method. In one embodiment the water-soluble conjugate is extracted by centrifugation. By “pre-treat” is meant that the composition is contacted or incubated with the reducing agent. In one embodiment the reducing agent is dithiothreitol, which can be used at any suitable concentration. For example, the pre-treatment can be with at least about 15 mg of dithiothreitol/100 ul, or at least about 10 mg/100 ul, or at least about 5 mg/100 ul, or at least about 20 mg/100 ul. Equivalent quantities of other reducing agents can also be used. The pre-treatment can be conducted for any suitable period of time, for example, 5 minutes or 10 minutes or 15 minutes, or 20 minutes, or longer than 20 minutes.

In another aspect, the present invention provides methods of preparing a water-soluble conjugate involving contacting a water-soluble intermediate precursor having at least one carrier and at least one linker, with at least one targeting element for a ligand to be detected or a ligand to be detected, and a non-specific protein to form a water-soluble intermediate conjugate. A signal component is attached to the water-soluble intermediate conjugate to form the water-soluble conjugate. A suspension is formed containing a precipitate of the water-soluble conjugate, and the water-soluble conjugate is extracted from the suspension. In various embodiments the non-specific protein can be bovine serum albumin, an immunoglobulin, or keyhole limpet hemocyanin. The carrier and linkers can also contain the signal component before addition of the targeting element or ligand, and non-specific protein, or the signal component can be added after attaching the targeting element or ligand and non-specific protein. In one embodiment the targeting element or ligand, and non-specific protein are contacted, added to, attached to, or incubated with the water-soluble intermediate precursor simultaneously. In one embodiment the water-soluble intermediate conjugate precursor consists of the carrier and the linker before addition of the targeting element or ligand. The targeting element or ligand and non-specific protein can be attached to the intermediate precursor at a salt concentration of at least 1.6 M or at least 1.7 M or at least 1.8 M or at least 1.9 M or at least 2.0 M or at least 2.2 M or at about 2.5 M. The non-specific protein can be reacted with the precursor at ratios of 1:1 or greater (precursor to non-specific protein) or 1:5 or greater, or 1:7 or greater, or 1:10 or greater, or 1:12 or greater, or 1:15 or greater, or 1:20 or greater. The method produces a water-soluble conjugate as described herein.

In another embodiment the conjugate can be created by attaching the non-specific protein to the carrier via the linker so that all linkers are blocked. The targeting element or ligand can then be attached to the precursor via the non-specific protein. The signal component can be attached either with the targeting element or ligand and non-specific protein, or can be attached in a later step to form the final conjugate.

In another aspect the present invention provides methods of preparing a water-soluble conjugate. The methods involve incubating a water-soluble conjugate precursor containing a carrier, a linker covalently bound to the carrier, and a signal component with a ligand to be detected or a targeting element for a ligand to be detected and a non-specific protein. A water-soluble conjugate having the non-specific protein covalently bound to the carrier via the linker is thus prepared. In one embodiment the non-specific protein is bound to the carrier via the linker, and to no other component of the water-soluble conjugate. The targeting element for a ligand to be detected (or a ligand to be detected) and the non-specific protein are incubated simultaneously with the precursor.

In another aspect the invention provides a device containing a water-soluble conjugate of the invention. The conjugate is located on a test strip, which is a porous carrier material having a sample zone and a detection zone. Liquid sample applied to the sample zone flows to the detection zone. The test strip also has a second targeting element selective for a targeting element bound to the ligand or for a ligand suspected to be present in the liquid sample, applied to the detection zone of the test strip. By “porous carrier” is meant a bibulous material through which fluid can move by capillary force. An example of such a material is nitrocellulose, although persons of ordinary skill in the art will identify other bibulous materials that also function in the invention, for example, polyamide, and pretreated papers. The “sample zone” is the area of the test strip where sample to be tested is applied. A “reagent zone” is an area where reagents are contained on the test strip. The reagents can be present in a dried form, and can be movably present on the strip. A “detection zone” is the area of the test strip where a measurement is taken to determine the presence, absence, or amount of a ligand suspected to be present in the sample. The device can also have a “label zone” where label is movably applied to the test strip. “Capillary force” refers to the interfacial forces that act among liquids in a capillary or in a porous medium, and which cause liquid to move through the capillary or porous medium. By “movably” is meant that the reagents or other composition can be moved along the device from one zone to another by the flow of liquid through the test strip.

Various embodiments of the devices can be provided. In one embodiment the water-soluble conjugate is in a dry form on the test strip, upstream of the detection zone and downstream of the sample zone. The test strip can also have a control zone, which can be located downstream from the detection zone. The “control zone” contains a targeting element, and binding at the control zone indicates that the assay is functioning as designed. In another embodiment the device also has a casing, which envelopes the test strip and defines the sample zone. The porous carrier can be backed with a moisture-impervious material, which is placed in contact with the inside of the casing. In one embodiment the device also has a cap, which is selectively received over one end of the casing and covers the sample zone of the device. The casing can be made of plastic or another suitable material. In one embodiment the test strip also contains a filter, which is situated upstream from the detection zone, and can be a part of the porous carrier material. The filter serves to remove any contaminating matter that may be present in the applied sample. The test strip can also be prepared so it has a portion of the binding sites within the test strip blocked with a blocking protein or polyvinyl alcohol. For example, the blocking protein can be bovine serum albumin, milk protein, or another material having an equivalent effect on the assay. The sample can be urine, serum, plasma, blood, semen, sputum, or another body fluid or other fluid of biological origin to be tested. In another embodiment the device has a test strip, a casing, and a portion of the test strip protrudes from the casing. For example, a 1 cm or less portion of the test strip protrudes from the casing for receiving sample.

In another aspect the present invention provides methods for preparing a water-soluble conjugate involving a) contacting a water-soluble intermediate precursor containing a carrier, a linker, a spacer, and a targeting element for a ligand to be detected or a ligand to be detected, with a signal component to form a suspension containing a precipitate comprising the water-soluble conjugate. Water-soluble conjugate is then extracted from the suspension.

In another aspect the present invention provides a water soluble conjugate containing at least one carrier component, at least one linking component, at least one electrochemical signal component covalently attached to the carrier component through the linking component; and at least one targeting element for a ligand to be detected or at least one ligand to be detected. The conjugate can also contain a spacer component, which can be attached to the carrier component through the linking component. By the electrochemical signal component being attached “through” the linking component is meant either that it is directly attached to the linking component without an intervening molecule, or that the signal component is attached to a component that is itself attached to the linking component. In one example, the signal component can be attached to the spacer component, which is attached to the linking component.

In one embodiment the electrochemical signal component is an enzyme that converts a substrate or a reaction mediator into an electrochemically detectable species. For example, the electrochemical signal component can be alkaline phosphatase, horseradish peroxidase, or another enzyme, and the substrate can be any that is appropriate for the enzyme, for example, 1-naphthyl phosphate, or hydroquinone, or other appropriate substrates for the enzymes. The electrochemically detectable species can therefore be 1-naphthol, or benzoquinone, or another species that results from the action of enzyme on the substrate used.

In one embodiment the electrochemical signal component is covalently attached to the spacer, and the ligand to be detected or targeting element for a ligand to be detected is covalently attached to the carrier through the linking component.

In another aspect the present invention provides methods of preparing a water-soluble conjugate. The methods involve contacting a carrier component bearing at least one linking component with at least one electrochemical signal component to form a water-soluble intermediate conjugate, and contacting the water-soluble intermediate conjugate with a ligand or targeting element for a ligand to be detected to form a water-soluble conjugate.

In different embodiments the molar ratio of carrier component to electrochemical signal component is 1:10 or greater, or 1:15 or greater, or about 1:20, or 1:20 or greater. In another embodiment the step of contacting of the water-soluble precursor with the targeting element to form the water-soluble conjugate results in the water-soluble conjugate being formed as a precipitate. The method can also include the step of subjecting the precipitate to sonication after forming the water-soluble conjugate.

In another aspect the present invention provides methods of detecting the presence or amount of an analyte in a sample. The methods involve contacting a surface coated with a specific binding molecule to the analyte to be detected with the sample, contacting the surface coated with the specific binding molecule to the analyte to be detected with a water-soluble conjugate as described herein, forming a binding complex of the specific binding molecule coated on the surface, the analyte, and the water-soluble conjugate, contacting the binding complex with a substrate for the electrochemical signal enzyme to form a product solution, and determining the presence or absence of analyte in the sample.

In one embodiment the specific binding molecule is an antibody or fragment thereof. The binding complex is the complex of the antibody attached to the surface, the analyte, and the conjugate. In various embodiments the surface can be an electrode, a magnetic bead, or another surface. When the surface is a magnetic bead, the method can further comprise contacting the product solution with an electrode to determine the presence or amount of analyte in the sample.

The summary of the invention described above is not limiting and other features and advantages of the invention will be apparent from the following detailed description, as well as from the claims.

### BRIEF DESCRIPTION OF THE DRAWINGS

FIG. 1 provides a graphical depiction of water-soluble conjugates and general techniques in their preparation to assist the reader in visualizing the conjugates and in following the procedures described in the specification.

FIG. 2 provides a comparison of responses of traditional HRP-dextran conjugate detection methods versus the present method.

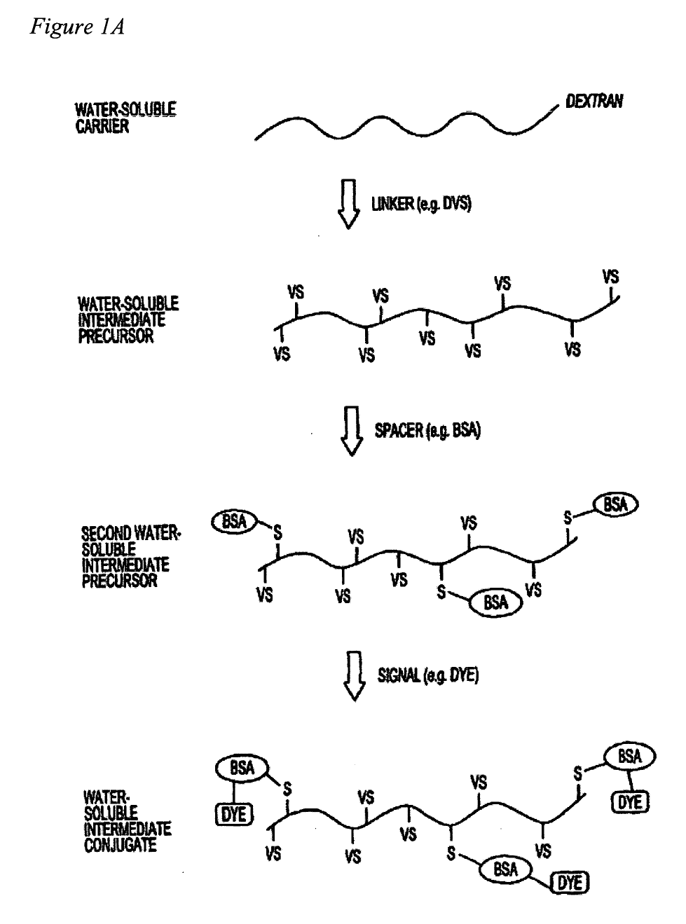

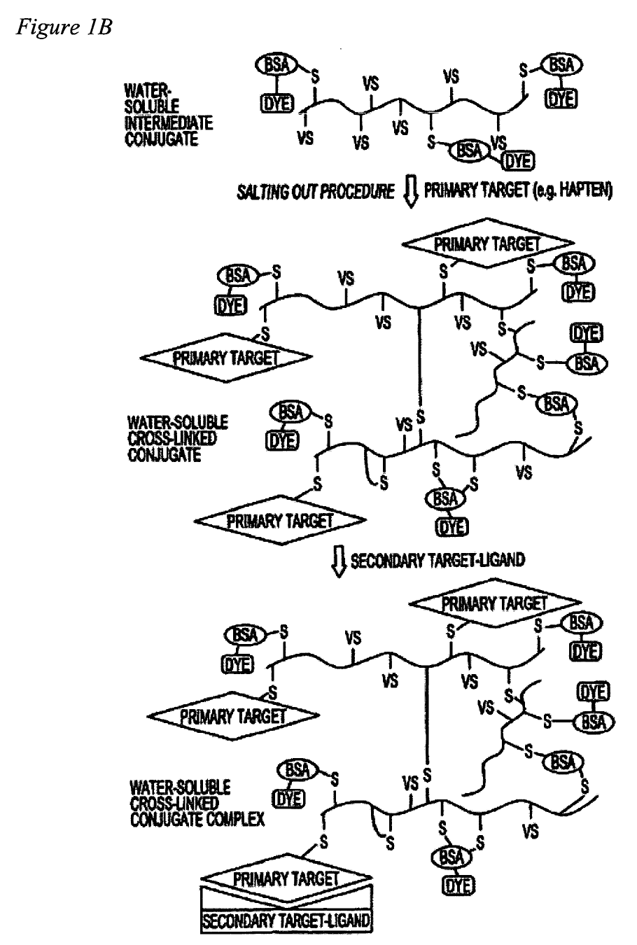

### DETAILED DESCRIPTION

The methods of the present invention allow higher product yields than previously have been obtainable by appropriate sonication of the water-soluble conjugate after cross-linking. The sonication process produces a clear solution, meaning that it contains no non-liquid matter visible with the unaided eye, or produces minimal non-liquid matter. Normally, after following this procedure, further centrifugation is unnecessary.

One aspect of the invention involves the washing of a pellet produced by centrifugation of a formed water-soluble conjugate, which is present as a precipitate in the reaction product. The supernatant is separated from the pellet and the pellet washed with an aqueous solution or buffer to form a second suspension. A second pellet is separated, and the wash step can be repeated 1-2 more times, as necessary. Without being bound by any particular theory, it is believed that the steps of washing the pellet solubilized free (unreacted) targeting element (e.g., an antibody), which is found in the supernatant. Thus, unreacted targeting element can then be easily disposed of. It was also found that the steps of washing eliminated the need to perform further purification of the product. Thus, the expensive step of purification of the product on, for example, a gel filtration (S-300) column was eliminated.

In another aspect the present invention provides water-soluble conjugates (and methods of their preparation) which contain a carrier, a linker, optionally a spacer component, a signal component, a targeting element for a ligand to be detected or a ligand to be detected, and a non-specific protein covalently bound to the carrier via the linker. Previously, large quantities of targeting element (or ligand) molecules were utilized to ensure sufficient coupling and cross-linking to produce the water-soluble conjugate. With the present invention, substantially less targeting element or ligand is necessary to ensure sufficient cross-linking than is used by binding to all available sites on the carrier. By including a non-specific protein in the reaction mixture, many sites on the carrier (available through the linker) will become occupied by the non-specific protein. Yet sufficient binding of targeting element or ligand will occur to result in a useful product. Thus, by utilizing the methods taught herein, the user is able to reduce the amount of targeting element (or ligand) utilized in the preparation procedure, and therefore substantially reduce the cost of producing the product.

In another aspect the present invention provides methods of preparing a water-soluble conjugate by contacting a water-soluble intermediate conjugate with a targeting element or ligand to form a suspension containing a precipitate that contains the water-soluble conjugate, extracting the water-soluble conjugate from the suspension, where the targeting element or ligand is pre-treated with a reducing agent prior to contact with the water-soluble intermediate conjugate. Pre-treatment of the targeting element with a reducing agent allows a higher binding rate of the targeting element to the carrier, resulting in an increase in the sensitivity of the assay. Example 5 provides a practical application of this aspect of the invention. Any reducing agent can be used, for example, dithiothreitol, beta-mercaptoethanol, Traut's Reagent (2-iminothiolane), or another reducing agent.

In another aspect the present invention provides methods for determining the presence an analyte in a liquid sample. The method involve contacting a liquid sample to a portion of the test device of the invention, the portion being located upstream from the detection zone; allowing the liquid sample to flow to the detection zone; and determining the presence, absence, or amount of the analyte in the liquid sample by observing the detection zone. The detecting step can be visually observing the signal in the detection zone.

The Carrier

The term “carrier” in the context of the present invention is used to denote the “backbone” of the conjugate, i.e. the carrier component functions as a backbone on which various components may be attached. The water-soluble polymers which function as the carrier component in the method for the preparation of conjugates may be chosen from a wide variety of types of polymers, including: natural and synthetic polysaccharides, as well as derivatives thereof, for example dextrans and dextran derivatives, starches and starch derivatives, cellulose derivatives, amylose and pectin, as well as certain natural gums and derivatives thereof, such as gum arabic and salts of alginic acid; homopoly(amino acid)s having suitable reactive functionalities, such as polylysines, polyhistidines or polyornithines; natural and synthetic polypeptides and proteins, such as bovine serum albumin (BSA) and other mammalian albumins; and synthetic polymers having nucleophilic functional groups, such as polyvinyl alcohols, polyallyl alcohol, polyethylene glycols and substituted polyacrylates.

Very suitable polymers for the purposes of the invention are polysaccharides and derivatives thereof, for example: dextrans, carboxymethyl-dextrans, hydroxyethyl- and hydroxypropyl-starches, glycogen, agarose derivatives, and hydroxyethyl- and hydroxypropyl-celluloses. As will be apparent from the working examples herein (vide infra), notably dextrans have proved to be particularly suitable polymers in connection with the invention.

It is often desirable, particularly for many of the immunochemical applications of the conjugates, that the conjugates have no, or substantially no, net charge, since the presence of a net positive or negative charge in such cases can lead, inter alia, to undesirable non-specific binding of the conjugates to substances and/or materials other than those of interest. In many cases this condition will, unless charged species are introduced, be fulfilled simply by ensuring that the polymeric carrier component itself possesses no net charge. Thus, a suitable polymeric carrier component for use in the method of the invention is, in its free state, substantially linear and substantially uncharged at a pH in the range of about 4 to about 10, the latter pH interval being the interval of practical relevance for the vast majority of immunochemical procedures, hybridization procedures and other applications of conjugates. Among various polymers which meet this criterion, are, for example, numerous polysaccharides and polysaccharide derivatives, e.g. dextrans and hydroxyethyl- and hydroxypropylcelluloses.

Depending on the use to which a conjugate is to be put, the conjugates may be based on water-soluble polymeric carrier components having a range of molecular weights in one embodiment of the invention, the polymeric carrier component may have a peak molecular weight in the range of about 40,000 to about 40,000,000 (prior to reacting the water-soluble polymeric carrier components with linker reagent such as DVS (divinyl sulfone) or EPCH (epichlorhydrin), or reacting resulting water-soluble intermediate precursor with a spacer or signal component for the eventual formation of cross-linked conjugate and cross-linked conjugate complexes). Peak molecular weights which are of considerable interest are peak molecular weights in the range of 100,000 to 10,000,000, such as in the range from 500,000 to 8,000,000, or in the range from 500,000 to 4,000,000, e.g. in the range from 500,000 to 2,000,000. Peak molecular weights of particular interest, notably in the case of dextrans as polymeric carrier components, are peak molecular weights of about 500,000, about 1,000,000, about 1,500,000, about 2,000,000, 2,500,000, about 3,000,000, about 3,500,000 and about 4,000,000.

More particularly, dextrans in the molecular weight ranges of 20,000 to 2,000,000 are suitable as starting carrier components. Most particularly, 20,000 Da dextrans are suitable for, but not restricted to, conjugates and/or complexes using streptavidin as the primary or secondary target. Furthermore, 500,000 Da dextrans are suitable for, but not restricted to, conjugates and/or complexes using certain dyes, enzymes, and with certain specific binding molecules as the primary or secondary target. Moreover, 2,000,000 Da dextrans are suitable for, but not restricted to, certain other dyes. In different embodiments the carrier can be any suitable carrier molecule, such as, for example, dextran, starch, glycogen, agarose, cellulose, natural gum, or mixtures thereof.

The term “peak molecular weight” as employed in the present specification and claims in connection with the carrier components denotes the molecular weight of greatest abundance, i.e. that molecular weight, among a distribution of molecular weights, which is possessed by the greatest number of molecules in a given sample or batch of the polymer. It is quite normal to characterize numerous types of polymers in this manner, owing to the difficulty (particularly for the highest molecular weights) of obtaining or preparing polymer fractions of very narrow molecular weight distribution. In the case of numerous commercially available carrier components which are of interest in the context of the invention, for example dextrans, the manufacturer or distributor will be able to provide reliable peak molecular weight data (determined, for example, by gel-permeation chromatography) which can provide a basis for the selection of the proper fraction of the polymeric carrier component. It should be mentioned here that peak molecular weight values (when used in connection with the carrier component) cited in the present specification and claims refer to the peak molecular weight of the free polymer in question, and take no account of, for example, the possible formation of cross-linked polymer units, e.g. as a result of cross-linking of two or more polymer molecules by reaction with a linking component such as DVS or EPCH during a method for the preparation of a conjugate; such cross-linked units will, on average, have higher molecular weights than the individual free polymer molecules from which they are formed.

The Linking Component

In the present context the terms “linker” or “linking component” are intended to cover bi-functional molecules capable of establishing covalent links between other—typically larger—molecules. Examples of linking components suitable for the method according to the invention are e.g. molecules comprising a bi-functional reactivity such as glutaraldehyde, carbodiimides, N,N′-phenylenedimaleimide, N-succinimidyl 3-(2-pyridylthio)propionate, p-benzoquinone, bis-oxiranes, divinyl sulfone (DVS) and epoxide derivatives, such as epoxides of the general formula I: 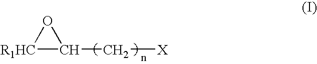
wherein R₁ is hydrogen or C₁₋₄-alkyl, n is an integer in the range from 1-4, i.e. 1, 2, 3 or 4, and X is a leaving group such as tosyl, mesyl, or halogen such as fluorine, chlorine, bromine, or iodine. In the present context the term “C₁₋₄-alkyl” designates a straight or branched saturated hydrocarbon group having from 1 to 4 carbon atoms, such as methyl ethyl, n-propyl, n-butyl, isopropyl, isobutyl, etc. As will be apparent from the working examples provided herein a very promising epoxide-derived linking component is epichlorohydrin (EPCH), i.e. a compound of the general formula I above, wherein R₁ is hydrogen, n is 1 and the leaving group X is chlorine.

The linking component should be stable in an aqueous environment and, accordingly, the linking component EPCH constitutes together with the linking component DVS a very useful linking components for use in the methods of the invention.

The Spacer Component

The spacer component is, via reaction with the linking component, covalently attached to the water-soluble intermediate precursor, thereby forming a second water-soluble intermediate precursor. As indicated above, the “spacer component” is covalently attached, via the linking group, to the carrier component. Thus, the term “spacer component” when used in the present context indicates a protein or a polypeptide which has a plurality of sites available for covalent attachment of signal components, such as dyes (vide infra). Spacer components are useful in any of the water-soluble conjugates used herein and in any of the methods of preparing water-soluble conjugates, although it is also possible for the conjugates to function without a spacer component.

One purpose for the incorporation of a spacer component, and particularly for a spacer having a plurality of sites available for covalent attachment of signal components, is that this method provides for a suitable means of increasing the number of signal components which can be attached to the conjugate (i.e. the “load” of the signal component in the water-soluble intermediate conjugate, vide ante), and thereby increasing the sensitivity of such conjugates when employed in various assays, e.g. immunochemical assays and in the lateral flow devices described herein (vide infra). It should be understood that in an embodiment wherein the coupling of a signal component (such as a dye molecule) is done directly to the linking component (and not through a spacer component) implies that (at least in principle) only one signal molecule is attached per molecule of linking component present in the conjugate.

In several embodiments of the preparation of the second water-soluble precursor, the number of moles of spacer per mole of starting dextran (the “load” of the spacer) ranges from 1 to 500, particularly from 2 to 100, most frequently from 5 to 75. Also, the second water-soluble intermediate can be characterized by, e.g., the number (moles) of spacer component attached per mole carrier component.

As stated earlier, only a fraction of the reactive moieties of the linking component of the water-soluble intermediate reacts with the spacer component. Depending on the spacer component and on the linker component, after reacting the spacer component, from 1 to 99% of the unreacted reactive moieties of the linker component, or 20-99%, particularly 30-99%, such as ranging from 40 to 99% and notably 50 to 99% remain unreacted. That is to say that, in one embodiment, under certain conditions, from 1 to 49% of the unreacted linker moieties reacted with the spacer component.

The spacer component can be a protein such as, for example, bovine serum albumin (BSA), ovalbumin, globulin, etc. or a polypeptide such as homopolypeptides, e.g. polylysines, polyhistidines, polyomithines, etc. However, the choice of spacer component will depend on the employed signal component (e.g. the actual dye employed in a particular conjugate) as well as the employed linking component.

The molecular weight of the spacer component, e.g. a protein, can be at least 2,500 Da, or at least 5,000 Da, or at least 10,000 Da, or in the range of 10,000-2,000,000, such as in the range of 20,000-500,000. As the one of the features of the introduced spacer components is to multiply the number of available positions for introduction of the signal components, it is furthermore desirable that the number of available functional groups for attachment of signal components is at least 5 per molecule of spacer component, for example, 10-1,000, in particular 10-500.

Alternatively, the spacer component can be a polysaccharide or polynucleic acid. Chemical modifications of these polymers may be required prior to the preparation of the water-soluble intermediate conjugate.

Owing to the nature of the coupling chemistry on the spacer component, (to both the linker component in the formation of the second water-soluble intermediate precursor, or later to a signal component in the formation of the water-soluble-intermediate conjugate, vide infra), a reactive functionality, such as a nucleophilic functionality, is present on the spacer component. Suitable spacer components will then be, for example, those with nucleophilic functional groups such as: —O⁻ (e.g. deprotonated phenolic hydroxy groups, such as deprotonated aromatic hydroxy groups in tyrosine residues of polypeptides or proteins), —S⁻ (e.g. deprotonated thiol groups on aromatic rings or aliphatic groups, such as deprotonated thiol groups in cysteine residues of polypeptides or proteins), —OH (e.g. aliphatic hydroxy groups present in certain amino acid residues of polypeptides or proteins, such as serine or threonine residues), —SH (e.g. thiol groups in cysteine residues of polypeptides or proteins), primary amino groups (e.g. in lysine or omithine residues of polypeptides or proteins) or secondary amino groups (e.g. in histidine residues of polypeptides or proteins). As will be understood by the skilled person, the question of whether the functional groups mentioned above will be in a protonated or de-protonated state will, of course, depend on the selected reaction conditions, such as the pH of the reaction mixture.

In one embodiment only a fraction of the unreacted reactive moieties of the linker component of the water-soluble intermediate react with the spacer component. That is to say that the second water-soluble intermediate still possesses a significant amount of unreacted reactive moieties.

The obtained second water-soluble intermediate precursor may be purified by the methods already discussed in connection with the purification step, i.e. in connection with the purification of the water-soluble intermediate precursor. As will be evident from the examples provided herein, a suitable method for purifying the obtained second water-soluble intermediate precursor is gel-filtration.

The Signal Component

In some embodiments the signal component is, via reaction with the spacer component, covalently attached to the second water-soluble intermediate precursor, thereby forming a water-soluble intermediate conjugate.

As used herein, the term “signal component” refers to moieties which are directly physically detectable or which are precursors for or produce such physically detectable moieties. In one embodiment the signal component functions as a label or a marker which can be readily measured by some physical technique known in art, e.g. by means of optical methods, such as spectrophotometry, fluorescence, luminescence, phosphorescence or other methods such as those described in e.g. “Instrumental Methods of Chemical Analysis” G. W. Ewing, 5th Ed., McGraw-Hill Book Company, New York, 1988. The signal component can also be detected by visual observation with the unaided eye. Alternatively, the signal component may—as indicated above—be a precursor for such a physically detectable component. A typical example of such a precursor is an enzyme which upon action on a suitable substrate is capable of generating species, for example colored species, which can be detected by one or more of the physical methods mentioned above.

The signal component may be selected from substances such as dyes; fluorescent, luminescent, phosphorescent and other light-emitting substances; metal-chelating substances, including iminodiacetic acid, ethylenediaminetetraacetic acid (EDTA), diethylene triaminepentaacetic acid (DTPA) and desferrioxamine B; substances labelled with a radioactive isotope; substances labelled with a heavy atom; and mixtures thereof

To give some further examples, fluorescent substances may be selected from, e.g., fluorescein (suitably as fluorescein isothiocyanate, FITC), fluoresceinamine, 1-naphthol, 2-naphthol, eosin, erythrosin, morin, o-phenylenediamine, rhodamine and 8-anilino-1-naphthalenesulfonic acid. Radioactive isotopes of relevance may be selected, for example, among isotopes of hydrogen (i.e. tritium, ³H), carbon (such as ¹⁴C), phosphorus (such as ³²P), sulfur (such as ³⁵S), iodine (such as ¹³¹I), bismuth (such as ²¹²Bi), yttrium (such as ⁹⁰Y), technetium (such as ^{99m}Tc), palladium (such as ¹⁰⁹Pd) and samarium (such as ¹⁵³Sm). Heavy atoms of relevance may be selected, for example, among Mn, Fe, Co, Ni, Cu, Zn, Ga, In, Ag, Au, Hg, I, Bi, Y, La, Ce, Eu and Gd. Gold (Au) is a particularly useful heavy atom in many cases.

In one embodiment signal components can be the non-particulate labels, for example, non-particulate dyes. In the present context the term “dye” is intended to mean any spectrophotometrically detectable dye molecule or derivative thereof. Dyes useful for incorporation into the conjugates prepared by the methods according to the invention include those derived from visual dyes, phosphorescent dyes, fluorescent dyes, laser dyes, infrared dyes and lanthanide chelates. Dyes which are particular interesting are visual dyes, including soluble visual dyes, such as pigments, vat dyes, sulphur dyes, mordant dyes, leucovat dyes and species such as fluorescein, rhodamine and derivatives thereof (such as sulphorhodamine, rhodamine-hydride and rhodamine hydrazide), as well as oxazine dyes, cyanine dyes and azol dyes. Specific examples of suitable dyes are, for example, Texas Red hydrazine, Congo Red, Trypan Blue, Lissamine Blue, Remazol Black, Remazol Brilliant Red, Rhodamine B Isothiocyanate, Cy5-Osu mono functional reactive dye, Reactive Orange 16, Uniblue A, etc. Dyes that are not particulate dyes are also useful in the present invention. A “non-particulate” label is one where the basis of detection of the label is other than the detection of a solid, whether the solid is the signal component (e.g., latex or other particles) or whether a solid precipitate is produced that is the basis for detection.

The above-mentioned dyes, which are useful as signal components for the purposes of the present invention, are all well-known in the art and it will be clear to the skilled person that other dyes can be used as signal components for the purposes of the present invention. Other examples of dyes to be used as signal components are e.g. such dyes as mentioned in “Dyeing and Chemical Technology of Textile Fibers”, Trotman, 34th Ed., C. Griffin & Co., London and “The Chemistry of Synthetic Dyes”, Vankataramon (Ed.), Academic Press, New York, 1979, the disclosures of which are incorporated herein by reference.

The signal component can be capable of reacting with a protein, such as BSA and/or, for alternative embodiments described below, capable of reacting with an unreacted reactive moiety of a linker component. Furthermore, the signal component, upon reacting or binding to the spacer, should not confer any undesirable properties of the resulting water-soluble intermediate conjugate, i.e. the signal component should not promote any uncontrollable non-specific binding nor inhibit the activity of the targeting components (e.g. antibodies) bound to the conjugate. Furthermore, the signal component should not reduce the water solubility of the conjugate significantly.

In one embodiment only a small fraction of the reactive moieties of the linking component of the second water-soluble intermediate reacts with the signal component in the formation of the water-soluble intermediate conjugate. Depending on the signal component, the spacer component, and on the linker component, after reacting the signal component, and relative to the amount of unreacted reactive linking component available in the second water-soluble intermediate precursor, from 50 to 100% of the unreacted reactive moieties of the linker component, for example 60-100%, particularly 70-100%, such as ranging from 80-100% and notably 90-100% remain unreacted (N.B. as compared to the second water-soluble intermediate precursor).

Depending on the particular dye, the conjugate prepared by the method of the invention reflects, scatters, or emits photons in the visible range, in the UV range or in the near infrared range. Use of a visual dye such as rhodamine will cause the conjugate of the invention to reflect or scatter photons in the visible region (e.g. blue), resulting in the transmission of the complementary wavelength of color (e.g. red) to an observer. Alternatively, the use of a fluorescent dye will (when radiated) cause the conjugate of the invention to emit photons at a specific wavelength due to the return of electrons to the ground state. A “visual dye” is a dye that reflects or scatters light in the visible range.

In one embodiment the signal component is a donor/acceptor dye pair. Donor/acceptor dye pairs are known in the chemical arts. In resonant energy transfer, the donor molecule absorbs a photon and initiates energy transfer to the acceptor. The acceptor receives the energy transfer and emits a photon. The donor dye and acceptor dye can perform fluorescent resonance energy transfer (FRET) upon excitation. Some examples of suitable donor/acceptor pairs are 6-carboxyfluorescein/6-carboxy-X-rhodamine (FAM-ROX), 3-(epsilon-carboxypentyl)-3′-ethyl-5,5′-dimethyloxacarbocyanine/6-carboxy-X-rhodamine (CYA-ROX), and the 4,4-difluoro-4-bora-3 alpha,4 alpha-diaza-s-indacene-3-propionic acid (BODIPY) derivatives, 5,7-dimethyl-BODIPY/5-(4-phenyl-1,3-butadienyl) BODIPY (BODIPY503/512-BODIPY581/591). These donor/acceptor pairs are provided by way of example and the person of ordinary skill with reference to the present disclosure will be able to identify more donor/acceptor dye pairs that exhibit FRET and are suitable use in the present invention. FRET is fluorescent resonance energy transfer, which is the transfer of the excited state energy from a donor to an acceptor.

In one embodiment, the spacer is covalently attached to the carrier via the linker, the signal component is a dye (e.g., a member of a donor/acceptor pair) covalently attached to the spacer, and the ligand or targeting element for a ligand is covalently attached to the carrier. The carrier is dextran, the linker is divinylsulfone, and the spacer is bovine serum albumin.

The methods are also suitable for the preparation of water-soluble conjugates wherein the signal component is covalently attached to the linking component, which in turn is attached to the carrier component, i.e. no protein or polypeptide spacer component is incorporated in the conjugate (vide infra). Further details are available in U.S. Pat. No. 6,627,460, column 12 inter alia.

In another embodiment the signal component is an electrochemical signal component, which can be covalently attached to the carrier through the linker. In one embodiment no spacer component is included in these conjugates, but the conjugate consists of a carrier molecule, linking component, and the electrochemical signal component bound to the linking component. The electrochemical signal component can be an enzyme that reacts with a substrate to produce an electrochemically detectable species. The electrochemically detectable species can be converted from the substrate, or can be a bi-product of the reaction, e.g., a reaction mediator. Examples of enzymes that can be used in the present invention include alkaline phosphatase, horseradish peroxidase, glucose 6-phosphate dehydrogenase, acetylcholinesterase, galactosidase, glucose oxidase, catalase, and choline oxidase. A combination of enzymes can also be used, whether combining enzymes that act independently, or a bi-enzymatic or multi-enzymatic system. One example of a bi-enzymatic system is NADH oxidase and alcohol dehydrogenase. Another bi-enzymatic system that can function in the present invention is tyrosinase and glucose dehydrogenase. Bi-enzymatic systems can use oxygen sensors for detection. When the electrochemical signal component is an enzyme (or combination of enzymes) it is termed an electrochemical signal enzyme.

Substrates or reaction mediators that can be used to generate the electrochemical signal include any substrate or reaction mediator that is appropriate for the enzyme selected, and which will produce an electrochemically detectable product or species. Examples of substrates include 4-aminophenyl phosphate, 1-naphthyl phosphate, glucose-6-phosphate, 4-hydroxynaphthyl-1-phosphate, 3-indoxyl phosphate, phenyl phosphate, 5-bromo-4-chloro-3-indolyl phosphate ester, 6-(N-ferrocenoylamino)-2,4-dimethylphenyl phosphate, paracetamol phosphate, 3,3′,5,5′-tetramethylbenzidine (TMB), hydroquinone, redox Os⁺²-based polymer, NAD⁺ and glucose-6-phosphate, acetylthiocholine iodide, 4-aminophenyl-beta-D-galactopyranoside (PAPG), glucose and a mediator, and choline. Examples of reaction mediators include ferricyanide, ferrocene, and ferrocene derivatives. These lists merely provide examples as many other substrates and reaction mediators can also be used. A “reaction mediator” is a substance that is distinct from the substrate and is utilized in the enzymatic reaction. Reaction mediators can be converted into electrochemically detectable species during the enzymatic reaction.

An “electrochemically detectable species” is a substance that is detectable by at least one of the electroanalytical techniques of voltammetry, potentiometry, or conductimetry. Examples of electrochemically detectable species include 4-aminophenol, 1-naphthol, glucose, dihydroxy naphthalene, indigo blue, phenol, H₂O₂, 6-(N-ferrocenoylamino)-2,4-dimethylphenol, 4-acetamidophenol (TMB ox), benzoquinone, ferricyanide, oxidized ferrocene and ferrocene derivatives, Os⁺³, NADH, thiocholine, 4-aminophenol (PAP), gluconolactone and a reduced mediator, and betaine. With reference to the present disclosure, the person of ordinary skill will realize many other enzymes, substrates, and electrochemically detectable species that can be used in the present invention.

Voltammetry is the electrochemical measuring technique used for electrochemical analysis or for the determination of the kinetics and mechanism of electrode reactions. In voltammetry the potential of the working electrode is controlled (typically with a potentiostat) and the current flowing through the electrode is measured. Potentiometry is the field of electroanalytical chemistry in which potential is measured under the conditions of no current flow. The measured potential may then be used to determine the analytical quantity of interest, generally the concentration of some component of the analyte solution. The potential that develops in the electrochemical cell is the result of the free energy change that would occur if the chemical phenomena were to proceed until the equilibrium condition has been satisfied. Conductimetry is the field of scientific measurement of solution conductance.

Ligands and Targeting Elements for a Ligand to be Detected

The term “targeting element” refers to molecules, especially molecules of biological origin, which are capable of binding to or reacting with, a complementary molecule or a complementary structural region of a material of biological origin. When the targeting element is a targeting element for the ligand to be detected, the targeting element binds to or reacts with the ligand to be detected.

Examples of relevant targeting elements for a ligand to be detected are, for example: monoclonal and polyclonal antibodies, gene probes, natural and synthetic oligo- and polynucleotides, natural and synthetic mono-oligo- and polysaccharides, lectins, avidin, streptavidin, biotin, growth factors, hormones, receptor molecules, protein A and protein G; and mixtures thereof. Particular examples include anti-human chorionic gonadotropin (anti hCG), luteinizing hormone (LH), Rabbit anti human CRP, streptavidin, avidin, anti-HIV, anti hepatitis C, anti Chlamydia, anti-herpes, anti-thyroid stimulating hormone (anti TSH), anti-Listeria, anti-salmonella, anti-mononucleosis, anti-HBeAb, anti-HBsAb, and anti-H. pylori.

Ligands to be Detected

“Ligands” are molecules to which targeting elements for the ligand will bind. Examples of ligands useful in the present invention are antigens and haptens, but can include any ligand of interest in the detection. Examples of hormones as ligands, are hormones (e.g. estrogen, estradiol, progesterone, human chorionic gonadotropin (hCG), luteinizing hormone, follicle stimulating hormone, cortisone, T3, T4), amino acid hormones (e.g. thyroxine) and peptide and protein hormones (e.g. vasopressin, bombesin, gastrin or insulin), and drugs of abuse. Other ligands include cardiac markers such as Troponin I, Troponin T, high sensitivity C-Reactive Protein (hsCRP), CK-MB, myoglobin, NT-proBNP, B-type natriuretic peptide (BNP), atrial natriuretic peptide (ANP), and myeloperoxidase; and cancer markers such as prostate specific antigen (PSA), carcinoembryonic antigen (CEA), and alpha-fetoprotein (AFP). A “drug of abuse” is a drug that is taken for non-medicinal reasons (usually for mind-altering effects). The abuse of such drugs can lead to physical and mental damage and (with some substances) dependence and addiction. Examples of drugs of abuse include cocaine, amphetamines (e.g., black beauties, white bennies, dextroamphetamines (dexies, beans), methamphetamines (crank, meth, crystal, speed)), barbiturates, lysergic acid diethylamide (LSD), depressants, sedatives (e.g., selective serotonin reuptake inhibitors), phencyclidine (PCP), tetrahydrocannabinol (THC), and opiates (e.g., morphine, opium, codeine, and heroin).

The present methods and compositions are useful in a variety of assay formats. For example, some formats utilize an antibody that is specific for the ligand suspected of being present in the sample. In these formats, reagents can be movably located on the test strip and sample is applied to the sample zone. Sample then migrates through the reagent zone where reagents can bind to ligand suspected of being present in the sample, and arrives at the detection zone where a targeting element is applied to the zone and binds to the ligand suspected to be present or the targeting element bound to the ligand, or even to another component of the conjugate. By “selective binding” is meant that the targeting element distinguishes between the ligand of interest from any other ligand likely to be present in the sample, so that the assay functions as intended. A targeting element that binds selectively can still bind to more than one ligand. By “specific binding” is meant that the targeting element binds to its target ligand and to no other ligand that may be present in the sample.

In another assay format two antibodies can be used which have a lower selectivity for the ligand of interest, and therefore can bind not only to the ligand of interest, but also to a second molecule present in the sample. In this format a scavenger antibody is used that binds to the binding sites on the second molecule, thus blocking these sites and leaving the two antibodies to bind only to the ligand of interest. In other formats, more than one scavenger antibody can be used and more than two antibodies can be used. With reference to the present disclosure the person of ordinary skill will be able to devise additional assay formats, which are also contemplated in the present invention. The particular formats listed herein are provided by way of example.

The invention can also be applied in a direct sandwich assay format. In this format, sample is applied to a sample zone, and flows through a label zone containing a label (e.g., dextran-BSA-anti-hCG Antibody-rhodamine) and if the ligand to be detected is present the label binds to the ligand. Sample then continues to flow to the detection zone (containing, for example, anti-hCG antibody affixed to the test strip). In the detection zone labeled ligand is bound to the detection zone and observation of the detection zone provides the result of the assay.

In another assay format (sometimes called an “indirect” format), sample is applied to a sample zone and migrates through a reagent zone containing a targeting element specific for the ligand to be detected movably present in the reagent zone (e.g. biotin-hCG Ab). The sample then continues to migrate through a label zone, which contains a conjugate of the invention that specifically binds to the ligand (e.g., the beta-portion of hCG) or to a targeting element bound to the ligand. Thus, a signal component is attached to the ligand to be detected. The sample then continues to migrate to the detection zone, where there is a bound a targeting element for the sample (e.g., streptavidin-IgG or streptavidin-BSA). Visual observation of the detection zone reveals the presence, absence, or amount of ligand present.

With reference to the present disclosure the person of ordinary skill in the art will realize other formats for application of the invention, which are also contemplated here. For example, the present invention can be applied in the formats described in the following references: U.S. Pat. Nos. 5,602,040, 5,622,871, 5,656,503, 6,187,598, 6,228,660, 6,352,862, 2001/0,008,774, 2001/0,041,368, 5,714,389, 5,989,921, 6,485,982, 5,252,496, 5,559,041, 5,728,587, 6,027,943, 6,506,612, 6,541,277, 2002/0,160,525, 5,073,484, 5,654,162, 6,020,147, 5,120,643, 5,578,577, 6,534,320, 4,703,017, 4,743,560, 5,591,645, 3,011,874, 3,641,235, 4,094,647, 4,168,146, 4,373,932, 4,477,575, 4,722,889, 4,861,711, 4,943,522, 4,978,503, 5,571,726, EP 0,149,168, EP 0,170,375, EP 0,192,320, EP 0,250,137, EP 0,287,731, EP 0,291,194, EP 0,349,215, GB 2,062,224, GB 8,709,873. The following examples are provided for further illustration of the invention.

Enzyme Electrochemical Immunoassay

In enzyme electrochemical immunoassay (ECI) the analyte or a second antibody is labeled with an enzyme that catalyzes the production of an electrochemically detectable product, and the rate of product formation used to quantitate the amount of analyte. Therefore, enzyme ECI utilizes antibody for specificity and the enzyme label for sensitivity through chemical amplification.

The majority of electrochemical detection techniques in immunoassay are based on voltammetry, the branch of electroanalysis that involves applying a potential to an electrochemical cell and measuring the current resulting from oxidation or reduction at the electrode. Of the many techniques of voltammetry, amperometry has been the most popular with ECI. In amperometry, the electrode is held at a fixed potential and the current produced from a redox event at the electrode surface is measured. Amperometry when applied to hydrodynamic electrochemical detectors yields detection limits at the nanomolar levels. Because detection can be done in very small sample volumes (less than 10 μL), the absolute amount of redox active species detected in a sample can be as low as 10⁻¹⁴ moles or less.

In amperometry, the optimum electrode potential for detection is chosen by obtaining the current response produced by the analyte as a function of the applied potential. This current response for an electroactive species in solution usually has three distinctive regions of behavior. One, a region of potential where the compound is not electroactive and current is negligible. Two, a region of rising current response defined by the Nernst equation. Third, a limiting current plateau that is independent of potential. The best potential for detection is along this limiting current plateau where analyte is being electrolyzed at the limit of mass transport to the electrode and small changes in the applied potential do not significantly affect the current measurement. The current response at the limiting plateau is directly proportional to the analyte concentration and is given by the equation I=nFADC^{o}/d, where I is the current, n the number of electrons involved in the redox reaction, F the Faraday constant, A the electrode surface area, D the diffusion coefficient, C^{o} the bulk analyte concentration, and d the thickness of the diffusion layer.

The Enzyme-Substrate-Product (E-S-P) System

The choice of the E-S-P system is an important factor in enzyme ECI. It is important that the enzymatic reaction be rapid and that the substrate S be redox inactive over some potential range where the product P is active. It is also preferable that the product P is electroactive at a low potential so that the background noise, which increases with rising potentials, remains low. A commonly used enzyme label alkaline phosphatase (ALP), with 4-aminophenyl phosphate (PAPP) as the substrate. Its enzymatic product 4-aminophenol (PAP) has a low oxidation potential (i.e., 0.18 V vs. Ag/AgCl at a glassy carbon electrode in pH 7 buffer) and causes no electrode poisoning. The less costly and more stable ALP substrate I-naphthyl phosphate has also been used. This E-S pair has shown to be especially good with screen-printed electrode based immunosensors. Other enzymes used in ECI are described herein.

Although the use of a single E-S pair is the most common practice in enzyme immunoassays, bi-enzymatic or multi-enzymatic systems can also be used. One detection scheme involves using tyrosinase to oxidize phenol first to catechol and then to O-quinone. O-quinone becomes a mediator in the enzymatic dehydrogenation of glucose and is re-converted to catechol. Quantitation of ALP is done indirectly by measuring the loss of O₂ in the oxidation of phenol.

Screen-printed electrodes (SPEs), fabricated with thick-film technology using a graphite powder-based ink to print electrodes on a polystyrene surface, and are adapted for immunosensors chiefly by passive adsorption of antibodies to the electrode surface. SPE-based immunosensors can be used in a number of applications for the assays described herein.

The compositions and methods of the invention can be used in any convenient assay format. However, two formats are well suited for electrochemical detection reagents. In one format, magnetic beads are treated with specific binding molecules (e.g., antibodies) to an analyte to be detected and placed into a vessel. A sample to be analyzed is contacted with the beads. If analyte is present in the sample it will be bound by the specific binding molecules on the beads. A composition of the present invention having an electrochemical signal component is added, and the mixture reacted. After appropriate rinsing, a substrate for the electrochemical signal component is placed into contact with the beads. This mixture can be reacted in a capillary tube for very small volumes. Very small volumes reduce the dilution of enzyme product, thus providing an enhanced electrochemical signal because the signal is linearly proportional to the concentration. After appropriate mixing and rinsing, a product solution is placed on an electrode and the presence or amount of analyte in the sample is determined.

In another embodiment specific binding molecules to an analyte are adsorbed to an electrode. In this type of assay, sample solution is placed in contact with the electrode. If analyte is present in the sample, it will be bound by the specific binding molecules. A composition of the present invention is placed into contact with the electrode to form a complex of the antibody to the analyte, the analyte, and the conjugate of the invention. After appropriate rinsing, a substrate for the electrochemical signal component is placed into contact with the electrode. After reaction, a reading is taken, for example using a voltammetry, potentiometry, or conductimetry, and the presence or amount of the analyte in the sample is determined.

By “specific-binding molecule” is meant a molecule that will selectively bind, through chemical or physical means to a detectable substance present in a sample. By “selectively bind” is meant that the molecule binds preferentially to the target of interest or binds with greater affinity to the target than to other molecules. In one embodiment the specific binding molecule is an antibody or a fragment of an antibody. “Antibody” refers to an immunoglobulin, whether natural or partially or wholly synthetically produced. The term also includes derivatives thereof which maintain specific binding ability. The term also covers any protein having a binding domain which is homologous or largely homologous to an immunoglobulin binding domain. These proteins may be derived from natural sources, or partly or wholly synthetically produced. An antibody may be monoclonal or polyclonal, and can be a member of any immunoglobulin class (or combination of classes), including any of the human classes: IgG, IgM, IgA, IgD, IgG, and IgE. An “antibody fragment” is any derivative of an antibody which is less than full-length. The antibody fragment can retain at least a significant portion of the full-length antibody's specific binding ability. Examples of antibody fragments include, but are not limited to, Fab, Fab′, F(ab′)₂, scFv, Fv, dsFv diabody, and Fd fragments. A “derivative” is any molecule having the same basic structure as the parent compound.

The antibody fragment may be produced by any means. For instance, the antibody fragment may be enzymatically or chemically produced by fragmentation of an intact antibody or it may be recombinantly produced from a gene encoding the partial antibody sequence. Alternatively, the antibody fragment may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

Single-chain Fvs (scFvs) are recombinant antibody fragments consisting of only the variable light chain (V_{L}) and variable heavy chain (V_{H}) covalently connected to one another by a polypeptide linker. Either V_{L} or V_{H} may be the NH₂-terminal domain. The polypeptide linker may be of variable length and composition so long as the two variable domains are bridged without serious steric interference. Typically, the linkers are comprised primarily of stretches of glycine and serine residues with some glutamic acid or lysine residues interspersed for solubility. “Diabodies” are dimeric scFvs. The components of diabodies typically have shorter peptide linkers than most scFvs and they show a preference for associating as dimers.

An “Fv” fragment consists of one V_{H} and one V_{L} domain held together by noncovalent interactions. The term “dsFv” is used herein to refer to an Fv with an engineered intermolecular disulfide bond to stabilize the V_{H}-V_{L} pair. A F(ab′)₂ fragment is an antibody fragment essentially equivalent to that obtained from immunoglobulins (typically IgG) by digestion with an enzyme pepsin at pH 4.0-4.5. The fragment may be recombinantly produced. A Fab′ fragment is an antibody fragment essentially equivalent to that obtained by reduction of the disulfide bridge or bridges joining the two heavy chain pieces in the F(ab′)₂ fragment. The Fab′ fragment may be recombinantly produced. A “Fab” fragment is an antibody fragment essentially equivalent to that obtained by digestion of immunoglobulins (typically IgG) with the enzyme papain. The Fab fragment may be recombinantly produced. The heavy chain segment of the Fab fragment is the Fd piece.

Active fragments of antibodies preferably include the Fv region of an antibody. Active fragments of antibodies can be made using methods known in the art, such as proteolytic digestion of samples including antibodies. Antibodies may be polyclonal or monoclonal, unless otherwise specified. A preparation of antibodies can be crude, such a whole blood or serum or plasma, or can be partially purified, such as by crude separation methods such as molecular weight purification or ammonium sulfate precipitation, or can be substantially purified, such as by affinity chromatography for a class of antibody, subclass of antibody, or by binding with a particular antigen or epitope. Methods for such purification are known in the art, such as provided by Harlow and Lane, Antibodies, A Laboratory Manual, Cold Spring Harbor Press (1988).

### EXAMPLE 1

### Preparation of Water-Soluble Conjugate

This embodiment of the preparation procedures involves four steps: activation of the dextran with divinyl sulfone, attaching BSA to the activated dextran, incorporating the rhodamine dye to the BSA portion of the dextran-BSA backbone, and cross-linking the antibody to the dextran-BSA-Rhodamine backbone.

Activated Dextran:

The following solutions were prepared for the activation: 25 mg/ml dextran (500,000 MW) in distilled water, 0.5 M potassium phosphate pH 11.4, and 25 mg/ml sodium borohydride in distilled water (prepared just prior to using).

The activation conditions were 10 mg/ml final concentration of dextran, 0.25 M potassium phosphate buffer, 0.25 mg/ml final concentration of sodium borohydride and 5% DVS. The entire operation was done in a fume hood. The dextran, distilled water and potassium phosphate buffer were combined initially and allowed to mix for 10-15 minutes. The sodium borohydride was added and followed immediately by the DVS. The timer was started from the first drop of DVS added and the DVS was added in a dropwise fashion within 2 minutes. After the entire amount of DVS was added, the solution was continually stirred for up to 30-35 minutes. After the 30-35 minute incubation, the activation was stopped by adjusting the pH to 7 with 25% HCl. The activated dextran was dialysed extensively against distilled water, with the water being changed twice a day for four days. The dialysate was collected, and chlorobutanol added at a final concentration of 0.01%.

Attachment of BSA to Activated Dextran:

The solutions prepared for the conjugation were: 50 mg/ml BSA (bovine serum albumin) in 0.1 M sodium chloride, 0.4 M potassium phosphate pH 10.4 and 0.1 M sodium chloride.

The conjugation conditions were: 1:25 activated dextran to BSA molar ratio, 0.010M K₂HPO₄, pH 10.4, 30° C., and 22 hours. The activated dextran, BSA solution and potassium phosphate buffer were added together. The pH of the mixture was adjusted to 10.4 with 1M HCl. The mixture was placed in a 30° C. oven for 22 hours. After the 22 hours incubation, the pH of the mixture was lowered to 6.5 with 1 M HCl. Then the mixture was purified using a S300 size exclusion column with 0.1 M sodium chloride as the running buffer. The first peak was collected and used for the next step.

Incorporation of the Rhodamine Dye to the BSA portion of the Dextran-BSA:

The following solutions were prepared: 1 M sodium bicarbonate pH 8.6, 10 mg/ml rhodamine isothiocyanate in DMSO, 0.5M K2HPO4 pH 7.2.

The conjugation conditions were 100-200 ug dye/mg BSA, 0.1 M sodium bicarbonate, pH 8.0, 30° C., and 1 hour. The dextran-BSA, rhodamine solution, and sodium bicarbonate buffer were added together. The pH was adjusted to 8.0 with 1 M HCl. The mixture was incubated in a 30° C. oven for 1 hour. After the incubation, the mixture was dialysed extensively against 10 mM K₂HPO₄ pH 7.2 (2 changes per day for 4 days). The dialysate was collected and Bronidox added at a final concentration of 0.05%.

Cross-Linking of Antibody to Dextran-BSA-Rhodamine:

The components needed for the cross-linking are: antibody solution, 3.5 M K₂HPO₄ pH 9-10, dextran-BSA-Rhodamine, 0.1 M cysteine in distilled water (prepared just prior to use), distilled water and 50 mM Tris pH 7.2/0.1M NaCl/0.02% sodium azide.

The cross-linking conditions were 1:2.5 to 1:5 dextran-BSA-rhodamine to antibody molar ratio, 30 ° C., 18-22 hours and 2.5 M K₂HPO₄ salt molarity. The dextran-BSA-rhodamine was centrifuged at 4000 g to remove any particulates. The antibody solution, dextran-BSA-rhodamine and K₂HPO₄ were combined together. The mixture was incubated in a 30° C. oven for 18-22 hours. After the incubation, cysteine was added at 1/10 the total volume. The salt concentration was adjusted from 2.5 M to 1.75 M by adding distilled water. Then the mixture was centrifuged at 9,333 g to pellet the water soluble conjugate. The pellet was resuspended in distilled water at ½ the original volume of dextran-BSA-rhodarnine used for the cross-linking. The resuspended pellet was centrifuged at 327 g for 5 minutes. The supernatant was purified in a S300 gel filtration column using 50 mM Tris/0.1M NaCl/0.02% sodium azide as the running buffer. The first peak was collected and used as a label conjugate.

### EXAMPLE 2

### Use of Sonication After Cross-Linking Antibody to Dextran-BSA-Rhodamine

The example illustrates the use of sonication in the methods. The cross-linking conditions were 1:2.5 dextran-BSA-Rhodamine to antibody molar ratio, 30° C., 18-22 hours and 2.5M salt molarity. The dextran-BSA-Rhodamine was centrifuged at 4000 g to remove any particulates. The antibody solution, dextran-BSA-Rhodamine and K₂HPO₄ were combined together. The mixture was incubated in a 30° C. oven for 18-22 hours. After the incubation, cysteine was added at 1/10 the total volume. The salt concentration was adjusted from 2.5. M to 1.75 M by adding distilled water. The mixture was then centrifuged at 9,333 g to pellet the water soluble conjugate. The pellet was resuspended in distilled water at ½ the original volume of dextran-BSA-Rhodamine used for the cross-linking. The resuspended pellet was sonicated (power set at 700 watts, 5 second/cycle, 10 cycles, 10 second pause between cycles) and then centrifuged at 327 g for 5 minutes. The supernatant was purified in a S300 gel filtration column using 50 mM Tris/0.1 M NaCl/0.02% sodium azide as the running buffer. The first peak was collected and used as a label conjugate.

For the preparation of a label pad, an OD 1.5 was used at 27-ul/test. Results showed a negative result when no ligand was present, and a positive result when 25 mIU/ml and 50 mIU/ml of ligand was present.

### EXAMPLE 3

### Use of Washing Procedure Eliminates Gel Filtration Column

The following procedure illustrates that washing of the pellet after precipitation eliminates the necessity of purification of the water-soluble conjugate by gel fitration or another step.

The following solutions containing antibody and “Dex-BSA-Rhodamine” were prepared: 0.00258 umole antibody and 0.00535 umole dextran (as “dex-BSA-rhodamine”) was mixed with 3.5 M potassium phosphate buffer, pH 11.5, to arrive at the following final concentrations: 2.5 M potassium phosphate buffer, pH 11.0. The molar ratio in the solution: “dex-BSA-rhodainine”/antibody was 1/2.5.

After mixing, a precipitate was observed in solution. Coupling was continued at 30 ° C. for 3 hours. After coupling, cysteine was added to the samples to a final concentration of 0.01 M cysteine. The concentration of phosphate buffer in solution was adjusted to 1.75 M by addition of de-ionized water to the solution. Solution was then spun for 5 minutes at 10,000 rpm and the supernatant, which was clear and almost colorless, was carefully aspirated with a pipette.

The precipitate (pellets) containing free antibody and coupled antibody was dissolved in 3 ml de-ionized water. The re-dissolved precipitate was spun for 10 minutes at 12000 rpm; the supernatant containing free antibody was discharged. The above step was repeated once. The precipitate (pellets) was then dissolved in 1 ml deionized (DI) water. The OD₅₅₈ of the “Dex-BSA-Rhodamine-Antibody” conjugates was measured and was greater than 20. Results are summarized below:

Results: OD_{558/280} = 41/39. Used OD = 20 to make label pad. Volume = 120 ul

| | 1 IUhCG/ml | 50 mIUhCG/ml | 25 mIUhCG/ml | Urine(−) | DI water |
|---|---|---|---|---|---|
| Sample-1 | +++ | ++ | + | − | − |
| Sample-2 | +++ | ++ | + | − | − |

### EXAMPLE 4

### Preparation of Water-Soluble Conjugate With Non-Specific Protein

This example illustrates the preparation of water-soluble conjugate using a non-specific protein, in this case BSA and immunoglobulin.

Method:
(1) The following materials were added in this order:
Monoclonal anti-beta HCG from Medix, clone 5008 10 mg
Dextran -BSA-Dye 6 ml (Dextran conc. 0.0043 um/ml), (Dex:Ab=1:2.5)
Mouse IgG from Acon Bio, R103008, 5 mg (Dex:mouse IgG=1:1.25) or without Mouse IgG
3.5 M K₂HPO₄ at pH 9.5 20.2 ml (final 2.5 M)
30 C, O/N
0.1 M cysteine 2 ml
DI water 6.67 ml
8000 rpm, 10 minutes
S-300 purification
Applied purified Ab conjugate to Label pad with OD 558=0.686, 59 ul per tests

Test Result:

| | With Mouse IgG | Without mouse IgG |
|---|---|---|
| Negative urine | − | − |
| hCG 25 mIU/ml | + | + |
| hCG 50 mIU/ml | + | + |

2) In a second example, the following materials were added in this order:

| | | | | | | | |
|---|---|---|---|---|---|---|---|
| 1 | 2 | 3 | 4 | 5 | 6 | 7 | 8 |

| | | | | |
|---|---|---|---|---|
| Ab | 5 mg | 5 mg | 2 mg | 1 mg |
| BSA | 0 | 4.224 mg | 6.8 mg | 7.66 mg |
| Dex-BSA- | 0.0128 um | 0.0128 um | 0.0128 um | 0.0128 um |
| rhodamine | | | | |
| Dex:Ab | 1:2.5 | 1:2.5 | 1:1 | 1:0.5 |
| Dex:BSA | 1:0 | 1:5 | 1:8 | 1:9 |

| | |
|---|---|
| K₂HPO₄ | 2.5M, pH 10 |
| Temperature | 30° C. |
| Time | 16 hours |

| | | | | |
|---|---|---|---|---|
| 10000 RPM, | Partial | soluble | soluble | soluble |
| 10 minutes | Soluble | | | |

| | |
|---|---|
| Purification | S-300 |

| | | | |
|---|---|---|---|
| Not | done | done | Lost |
| finished | | | |

Applied purified Ab conjugate to Label pad with OD 558=0.686, 59 ul per tests

| | With BSA (1:5) | With BSA (1:8) |
|---|---|---|
| Negative urine | − | − |
| HCG 25 mIU/ml | + | + |
| HCG 50 mIU/ml | + | + |

### EXAMPLE 5

### Preparation of Water-Soluble Conjugate by Pre-Treatment with Dithiothreitol

This example illustrates the preparation of a water-soluble conjugate using pre-treatment of antibody (targeting element) with dithiothreitol.

Dex-BSA-Rhodamine, dex conc. 0.00464 uM/ml, Ab: Monoclonal anti-beta hCG, clone 5008, 4.8 mg/ml.

2. Method

| | 1 | 2 | 3a | 3b | 4 |
|---|---|---|---|---|---|
| Ab | 4 mg | 4 mg | 4 mg | 4 mg | 2 mg |
| DTT | 17 ul | 34 ul | 80 ul | 80 ul | 0 |
| 15.4 mg/100 ul | | | | | |
| RT | 30′ | 1 h15′ | 1 h30′ | 1 h30′ | no |
| Purification | Yes | Yes | Yes | Yes | no |
| with Column | | | | | |
| PD10 | | | | | |
| Dex-BSA- | 1.1123 | 1.1123 ml | 1.1123 ml | 1.1123 ml | 1.1123 ml |
| Dye | ml | | | | |
| Dex:Ab | 1:5 | 1:5 | 1:5 | 1:5 | 1:2.5 |
| 3.5MK₂HPO₄ | 7.03 ml | 6.53 ml | 6.52 ml | 6.52 ml | 3.8225 ml |
| | (2.5M) | (≈2.3M) | (≈2.3M) | (2.5M) | (2.5M) |
| Buffer pH | 9 | 9 | 9 | 8.5 | 9 |
| 30 C. 11.5 h | Yes | Yes | Yes | Yes | Yes |
| Cystein 1/10 | Yes | Yes | Yes | Yes | Yes |
| volume | | | | | |
| DI water to | 1.75 M | 1.75 M | 1.75 M | 1.75 M | 1.75 M |
| final salt | | | | | |
| Conc. | | | | | |
| 10000 RPM, | Precipi- | Precipitate | Precipitate | Precipitate | Precipitate |
| 10 minutes | tate | | | | |
| D.I water | 0.6 ml | 0.6 ml | 0.6 ml | 0.6 ml | 0.6 ml |
| 3000 RPM × 5 | Most | soluble | soluble | soluble | soluble |
| minutes | precipi- | | | | |
| | tate | | | | |
| S-300 | Yes | Yes | Yes | Yes | Yes |
| purification | | | | | |
| Label pad | | | | | |
| OD1.5/ | | | | | |
| 27 ul/test | | | | | |

Result

NC: FHC 102 stripped with Monoclonal anti alpha hCG, Acon Bio

Sample

| | LP 2 | LP 3a | LP 3b | LP 4 |
|---|---|---|---|---|
| DI water | − | − | − | − |
| Negative urine | − | − | − | − |
| hCG 25 mIU/ml | + (L3) | + (L3) | + (L4) | + (L5) |
| hCG 50 mIU/ml | + (L4) | + (L4) | + (L4) | + (6) |
| hCG 100 mIU/ml | + | + | + | + |
| hCG 1 IU/ml | + | + | + | + |

### EXAMPLE 6

### Alternate Stepwise Conjugates

This example illustrates one alternate method of preparing the water-soluble conjugate. In this method, the signal component is linked to the targeting element prior to the combination with the water-soluble intermediate conjugate to form the water-soluble conjugate.

The bovine serum albumin was conjugated to activated dextran. The composition was purified to separate the free BSA. The hCG antibody was then conjugated to dextran-BSA at a molar ratio of 5:1 in 0.1 M potassium phosphate, pH 9.6, at 30 ° C. for 18 hours. The composition was again purified to separate the free antibody. Rhodamine dye was conjugated to the dextran-BSA-antibody at a ratio of 150 ug dye/mg protein in 0.1 M sodium bicarbonate, pH 8.0 at 30 ° C. for 3 hours. The reaction was stopped with cysteine and dialysed extensively again 10 mM K₂HPO₄ pH 7.2. Lastly, the antibody was cross-linked to the dextran-BSA-antibody-dye at a ratio of 2.5:1 in 2.5 M K₂HPO₄, pH 10.6 at 30° C. for 18 hours. The conjugate was then purified to separate the free antibody.

An OD of 0.8 at 27 ul/test was used to make the label pad. Results showed a negative result when no ligand was present, and a positive result when 100 mIU/ml of ligand was present.

### EXAMPLE 7

### Indirect Assay Format

This example illustrates the use of the invention in an indirect assay format. The water soluble conjugate was prepared according to the procedures described above, except that after the first centrifugation, the pellet was washed three times in distilled water. The final pellet was then resuspended in distilled water. The solution was sonicated using 5-second cycles for 10 cycles with 10-sec pause between cycles. A label pad was made at OD₅₅₀ 45.

The label pad was evaluated according to this configuration: a test strip containing a sample zone, a biotinylated alpha-hCG antibody in the reagent zone, a label pad, and Streptavidin-IgG striped down on the nitrocellulose and absorbent in the detection zone. The test strip was placed inside a plastic housing. The test device was tested with different levels of hCG concentration, negative urine and distilled water.

The results obtained at 3 minutes were negative for distilled water and urine with no hCG. And a positive result was obtained for samples containing 1 IU/ml, 500 mIU/ml, 100 mIU/ml, and 50 mIU/ml.

### EXAMPLE 8

### Conjugation of HRP to Activated Dextran

This example illustrates the conjugation of HRP to DVS-activated dextran of MW 500,000. Activated dextran with a molecular weight of 500,000 with an extent of activation of 26% was added to HRP solution, in a molar ratio of 1:20 (dextran:HRP). The coupling buffer was 10 mM phosphate, pH 10.4. Coupling continued for 22 hours at 30° C. using HRP in 40 mg/ml. After removal of the bottle from the 30 ° C. incubator, the pH was titrated to 6.5 with 1 M HCl. The solution was separated on a Sephacryl® S-200 gel filtration column. The column was equilibrated in 0.1 M NaCl, which was degassed before use. The purification was performed by isocratic elution with 0.1 M NaCl as eluent. HRP elution was detected at 403 nm.

### EXAMPLE 9

### Production of Anti-hCG Conjugate for Electrochemical Immunoassay

This example illustrates the synthesis of an anti-hCG conjugate by conjugation of an anti-hCG antibody to divinyl sulfone-activated dextran-HRP, utilizing precipitation in a high salt buffer.

Anti-beta-hCG (R006003, 6.5 mg/ml in PBS) is conjugated to an activated dextran-HRP-conjugate via free divinylsulfone (DVS) groups on the dextran. After coupling and precipitation, any free un-reacted VS groups are blocked by addition of cysteine. The precipitate is pelleted by centrifugation and resuspended by sonication in deionized water. The dextran-HRP/anti-hCG conjugate is separated from unbound antibody by gel filtration on Sephacryle S-300. The purified conjugate is measured at 280 nm.

A molar ratio of 2 moles of antibody per 1 mole of dextran-HRP was used. 0.46 ml of Dextran-HRP with 0.95 ml of anti-beta-hCG (6.5 mg/ml) in phosphate-buffered saline (PBS). 1.9 ml of K₂HPO₄ (3 M, pH 9.0) was added dropwise to bring the phosphate concentration to 2 M, while swirling in a 15 ml conical tube. The total volume was 3.31 ml. The tube was incubated with mild shaking at 125 rpm at 30 ° C. for 18 hours. After 18 hours the precipitate is accumulated near the solution surface. 0.46 ml of deionized water was added, and the solution mixed to adjust the phosphate concentration to 1.75 M. 0.377 ml of 0.1 M cysteine was added to cap any remaining vinyl groups. The solution was then mixed gently and allowed to stand for 15 minutes at room temperature. The mixture was transferred to 2 ml microfuge tubes and spun for 15 minutes at 10,000 rpm. The clear supernatant was removed and the pellet resuspended and combined into a volume of about 1.5 ml of deionized water.

The tube was sonicated in a cuphorn sonicator, maintaining ice in the cuphom bath. The controller of the sonicator was set for 5 second pulses, 90% maximum output. The polypropylene plastic tube was pressed against the sonic probe during the sonication to achieve maximum energy transfer at 4° C. Sonication was performed in 3 minute intervals. The tube remained on ice and was pressed against the sonicator four times for the 3 minute intervals. This procedure was found to discourage micro-heating of sample and to maximize re-solubilization of sample.

The pellet was again spun down and the supernatant was saved. The pellet was resuspended in 0.35 ml of DI water and the sonication procedure repeated. The cycles of sonication and spinning were repeated until at least 90% of the pellet was dissolved. The solutions were then combined.

The dissolved conjugate was concentrated to a volume of approximately 1 ml using a spin concentrator with a molecular weight cutoff of 30,000. The conjugate was then applied to an S-300 column (at least 31 ml of bed volume), pre-equilibrated with 50 mM Tris, 0.1 M NaCl, pH 7.2. Elution was at a flow rate of 1 ml/min with Tris buffered saline, and the first peak was collected into one or two fractions. The conjugate eluted in the first peak, and peak fractions were combined.

### EXAMPLE 10

### Coating of Electrode With Antibody for Electrochemical Detection

This example illustrates the preparation of antibody coated carbon electrodes to be used as an affinity sensor for electrochemical detection of hCG.

A screen-printed carbon electrode was used having a Melinex® ST328 polyester film substrate. The electrodes were printed using graphite ink and silver chloride ink. The screen printer was an SMT Optiprint, model 1616, PD-F.

The electrode was soaked in coating buffer with 100 μg/ml anti-α hCG antibody for 2 hours at room temperature, followed by soaking in a blocking buffer for 1 hour at room temperature. After washing and drying, the electrodes were stored dessicated.

The coated electrodes were incubated in sample matrix or PBS buffer containing known concentrations of hCG, such as 0, 2, 5, or 50 mIU/ml hCG for 30 minutes at room temperature. After washing, the electrodes were soaked in anti-β hCG label conjugate (3 μg/ml) in label conjugate buffer for 30 minutes at room temperature. After washing, 20 μl of substrate was applied to the electrodes (20 mM naphthol phosphate, 0.1 M NaCl, 0.1 M diethanolamine pH 9.6, for ALP conjugate), and incubated for 10 minutes at room temperature. Signal was then recorded using differential pulse voltammetry.

### EXAMPLE 11

### Comparison of Traditional HRP-Ab Detection v. HRP-Dextran-Ab

The capture surface was prepared by mixing 50 ul of Dynabeads® M-280 Streptavidin and 133 ul of 100 ug/ml biotinylated 6002 hCG capture antibody to a vessel, and mixing for 50 minutes. The beads were washed with Rinse Buffer (80 ul of phosphate buffer (pH 7.2) with 0.5% BSA and 0.5% Tween). 80 ul of 1% casein was added and the mixture incubated for 2 hours. The mixture was then washed with 1% casein, and re-suspended in 166 ul of 1% casein, and placed in the refrigerator at 4° C.

Two tubes were labeled A and B. To tube A was added 10 ul of the beads. After suitable rinsing 5 ul of HRP-6003 conjugate prepared as described herein was padded and the mixture placed on a shaker. 50 ul of hCG standard was added to Tube A while mixing. The mixture was incubated for 7.5 minutes, and wash twice with Rinse Buffer (as above), and re-suspended in 50 ul of Rinse Buffer.

The beads in Tube A were transferred to Tube B, and washed with phosphate-buffered saline (PBS). The beads were then separated with a magnet and the supernatant removed. After washing 15 ul of enzyme substrate mixture (10 mM hydroquinone with freshly mixed H₂O₂ to give a final concentration of 10 mM), mixing for 20 minutes was performed. The beads were separated, and 5 ul of beads were pipetted onto an electrode to run differential pulse voltammetry.

For comparison, in a separate experiment the capture surface was prepared by mixing 30 ul of Dynabeads® M-280 Streptavidin with 80 ul of 100 ug/ml biot-6002 antibody. The mixture was mixed for 45 minutes, and washed 1× with 80 ul of phosphate buffer (pH 7.2) with 0.5% BSA and 0.5% Tween. 80 ul of 1% casein was added and incubated for 2 hours. The mixture was washed with 80 ul of casein, and re-suspended in 100 ul of 1% casein. The labeled antibody was alkaline phosphatase-6003 conjugate antibody prepared as described herein. hCG standards were prepared as follows:
Well 400—add 120 ul of 1% casein+80 ul hCG
Well 200—add 100 ul of 1% casein+100 ul from above tube
Well 100—add 100 ul of 1% casein+100 ul from above tube
Well 50—add 100 ul of 1% casein+100 ul from above tube
Well 25—add 100 ul of 1% casein+100 ul from above tube
Well 12.5—add 100 ul of 1% casein+100 ul from above tube
Well 0—add 100 ul 1% casein

The hCG standards were prepared along one column of a micro-titer plate, and 5 ul of labeled antibody per well was added. At 2 minutes time, 50 ul of hCG standards was rapidly transferred from column 12 to 1, and mixed and pre-incubated for 2 minutes. 10 ul of beads was added per well while mixing on a plate shaker, and incubated for 8 minutes. The beads were separated with a magnet and the supernatant was removed from each well. The beads were rinsed 2× with 70 ul of phosphate buffer (pH 7.2) with 0.5% BSA and 0.5% Tween. 70 ul of the phosphate buffer was then added to each well, and the beads transferred to adjacent wells in column 2. The beads were again rinsed with 70 ul of the phosphate buffer. 15 ul of substrate (20 mM 1-naphthyl phosphate in 100 mM diethanolamine, pH 9.6) was added, and the mixture incubated for 25 minutes. The beads were held and 8 ul of substrate was transferred directly onto a screen printed electrode for differential pulse voltammetric measurements.

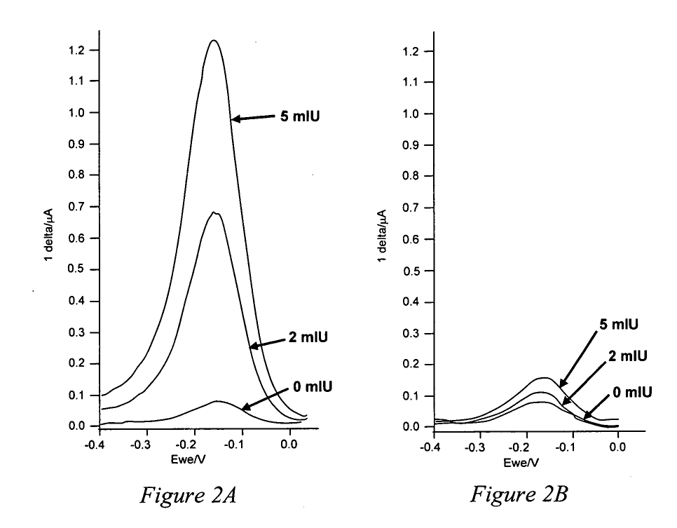

FIG. 2 shows that, when electrochemical detection was utilized according to the present invention, a several-fold higher sensitivity was obtained compared to traditional electrochemical methods. When one corrects for the nearly twice as long incubation time (20 min) used in the traditional methods versus 11 minutes for the present method, the difference is even more dramatic.

The invention illustratively described herein may be practiced in the absence of any element or elements, limitation or limitations that are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by various embodiments and optional features, modification and variation of the concepts herein disclosed may be resorted to by those skilled in the art, and that such modifications and variations are considered to be within the scope of this invention as defined by the appended claims.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other documents.

1. A water soluble conjugate comprising:
at least one carrier component;
at least one linking component;
at least one electrochemical signal component covalently attached to the carrier component through the linking component;
at least one targeting element for a ligand to be detected or at least one ligand to be detected.

2. The water-soluble conjugate of claim 1 further comprising a spacer component, which is attached to the carrier component through the linking component.

3. The water-soluble conjugate of claim 2 wherein the spacer is selected from the group consisting of: a protein, a polypeptide, bovine serum albumin, ovalbumin, or a globulin.

4. The water-soluble conjugate of claim 1 wherein the carrier is selected from the group consisting of: dextran, starch, glycogen, agarose, cellulose, natural gum, and mixtures thereof; and
the at least one targeting element for a ligand to be detected or at least one ligand to be detected is attached to the carrier through the linking component.

5. The water-soluble conjugate of claim 4, wherein the carrier is dextran.

6. The water-soluble conjugate of claim 1 wherein the linking component is divinyl sulfone.

7. The water-soluble conjugate of claim 1 wherein the electrochemical signal component is an enzyme that converts a substrate or a reaction mediator into an electrochemically detectable species.

8. The water-soluble conjugate of claim 7 wherein the enzyme is selected from the group consisting of: alkaline phosphatase, horseradish peroxidase, beta-galactosidase, acetylcholinesterase, choline oxidase, glucose oxidase, catalase, and glucose-6-phosphate dehydrogenase.

9. The water-soluble conjugate of claim 8 wherein the substrate or reaction mediator is selected from the group consisting of: 4-aminophenyl phosphate, 1-naphthyl phosphate, glucose-6-phosphate, 4-hydroxynaphthyl-12-phosphate, 3-indoxyl phosphate, phenyl phosphate, 5-bromo-4-hloro-3-indolyl phosphate ester, 6-(N-ferrocenoylamino)-2,4-dimethylphenyl phosphate, paracetamol phosphate, 3,3′,5,5′-tetra-methylbenzidine (TMB), hydroquinone, NAD⁺ and glucose-6-phosphate, acetylthiocholine iodide, 4-aminophenyl-beta-D-galactopyranoside (PAPG), glucose, and choline; and
the electrochemically detectable species is selected from the group consisting of: 1-naphthol and benzoquinone.

10. The water-soluble conjugate of claim 2 wherein
the electrochemical signal component is covalently attached to the spacer; and
the ligand to be detected or targeting element for a ligand to be detected is covalently attached to the carrier through the linking component.

11. The water-soluble conjugate of claim 1 wherein the targeting element for a ligand to be detected is selected from the group consisting of: a monoclonal or polyclonal antibody, a receptor molecule, a lectin, avidin, streptavidin, and biotin.

12. The water-soluble conjugate of claim 1 wherein the ligand is selected from the group consisting of: an antigen, a hapten, a gene probe, an oligo- or polynucleotide, a mono-, oligo- or polysaccharide, a hormone, a pharmacological agent, a peptide, a lectin, avidin, streptavidin, biotin, a growth factor, and a protein.

13. The water-soluble conjugate of claim 1 wherein the ligand is selected from the group consisting of: a bacterial or viral antigen, human chorionic gonadotropin (hCG), luteinizing hormone (LH), follicle stimulating hormone, cortisone, T3, T4, a drug of abuse, Troponin I, Troponin T, high sensitivity C-reactive protein (hsCRP), CK-MB, myoglobin, NT-proBNP, B-type natriuretic peptide (BNP), atrial natriuretic peptide (ANP), prostate specific antigen (PSA), carcinoembryonic antigen (CEA), and alpha-fetoprotein (AFP).

14. A method of preparing a water-soluble conjugate comprising:
contacting a carrier component bearing at least one linking component with at least one electrochemical signal component to form a water-soluble intermediate conjugate;
contacting the water-soluble intermediate conjugate with a ligand or targeting element for a ligand to be detected to form a water-soluble conjugate.

15. The method of claim 14 wherein the molar ratio of carrier component to electrochemical signal component is 1:10 or greater.

16. The method of claim 14 wherein the carrier component is dextran and the electrochemical signal component is horseradish peroxidase.

17. The method of claim 14 wherein the step of contacting the water-soluble intermediate conjugate with the targeting element to form the water-soluble conjugate results in the water-soluble conjugate being formed in the form of a precipitate.

18. The method of claim 17 further comprising subjecting the precipitate to sonication after forming the water-soluble conjugate.

19. The method of claim 14 wherein the carrier is selected from the group consisting of: dextran, starch, glycogen, agarose, cellulose, natural gum, and mixtures thereof.

20. The method of claim 14 wherein the linking component is divinyl sulfone.

21. The method of claim 14 wherein the electrochemical signal component is an enzyme that converts a substrate or reaction mediator into an electrochemically detectable species.

22. The method claim 21 wherein the enzyme is selected from the group consisting of: alkaline phosphatase, horseradish peroxidase, beta-galactosidase, acetylcholinesterase, choline oxidase, catalase, and glucose-6-phosphate dehydrogenase.

23. The method of claim 22 wherein the substrate or reaction mediator is selected from the group consisting of: 4-aminophenyl phosphate, 1-naphthyl phosphate, glucose-6-phosphate, 4-hydroxynaphthyl-12-phosphate, 3-indoxyl phosphate, phenyl phosphate, 5-bromo-4-chloro-3-indolyl phosphate ester, 6-(N-ferrocenoylamino)-2,4-dimethylphenyl phosphate, paracetamol phosphate, 3,3′,5,5′-tetra-methylbenzidine (TMB), hydroquinone, NAD⁺ and glucose-6-phosphate, acetylthiocholine iodide, 4-aminophenyl-beta-D-galactopyranoside (PAPG), glucose, choline, ferrocene, and ferricyanide; and
the electrochemically detectable species is selected from the group consisting of: 4-aminophenol, 1-napthol, benzoquinone, ferricyanide, and thiocholine.

24. The method of claim 14 wherein the targeting element for a ligand to be detected is selected from the group consisting of: a monoclonal or polyclonal antibody, a receptor molecule, a lectin, avidin, streptavidin, and biotin.

25. The method of claim 14 wherein the ligand is selected from the group consisting of: an antigen, a hapten, a gene probe, an oligo- or polynucleotide, a mono-, oligo- or polysaccharide, a hormone, a pharmacological agent, a peptide, a lectin, avidin, streptavidin, biotin, a growth factor, a protein, or mixtures thereof.

26. A method of detecting the presence or amount of an analyte in a sample comprising:
contacting a surface coated with a specific binding molecule to the analyte to be detected with the sample;
contacting the surface coated with the specific binding molecule to the analyte to be detected with a water-soluble conjugate comprising:
at least one carrier component;
at least one linking component;
at least one electrochemical signal enzyme covalently attached to the carrier component;
at least one targeting element for the analyte to be detected; and
forming a binding complex of the specific binding molecule coated on the surface, the analyte, and the water-soluble conjugate;
contacting the binding complex with a substrate for the electrochemical signal enzyme to form a product solution;
determining the presence or absence of analyte in the sample.

27. The method of claim 26 wherein
the specific binding molecule is an antibody or fragment thereof;
the electrochemical signal enzyme is selected from the group consisting of: alkaline phosphatase and horseradish peroxidase; and
the substrate is selected from the group consisting of: 1-naphthyl phosphate and hydroquinone.

28. The method of claim 27 wherein the surface is a magnetic bead.

29. The method of claim 28 further comprising contacting the product solution with an electrode.

30. The method of claim 27 wherein the surface is an electrode.
